(19) United States
(12) Patent Application Publication
(10) Pub. No.: US 2022/0024960 A1
(43) Pub. Date: Jan. 27, 2022

(54) IMPROVED SYNTHESIS OF AN EXPOXIDATION-CATALYST

(71) Applicant: DSM IP Assets B.V., Heerlen (NL)

(72) Inventors: Werner BONRATH, Kaiseraugst (CH); Rolf KUENZI, Kaiseraugst (CH)

(21) Appl. No.: 17/414,029

(22) PCT Filed: Dec. 17, 2019
(86) PCT No.: PCT/EP2019/085495

§ 371 Date: Jun. 15, 2021

(30) Foreign Application Priority Data
Dec. 20, 2018 (EP) 18214438.6

(51) Int. Cl. C07H 3/02; C07H 1/00; B01J 31/02

(52) U.S. Cl. CPC C07H 3/02; B01J 31/0208; C07H 1/00

(57) ABSTRACT

The present invention relates to an improved process to produce a specific and very efficient epoxidation-catalyst (1,2:4,5-Di-O-isopropylidene-β-D-erythro-2,3-hexodiulo-2,6-pyranose).

The present invention relates to an improved process to produce a specific and very efficient epoxidation-catalyst (1,2:4,5-Di-O-isopropylidene-β-D-erythro-2,3-hexodiulo-2,6-pyranose).

1,2:4,5-Di-O-isopropylidene-β-D-erythro-2,3-hexodiulo-2,6-pyranose (also known as Shi catalyst) was developed by Yian Shi and his team. It was first published in 1996 (J. Am. Chem. Soc. 1996, 118, 9806-9807).

The Shi catalyst has the following chemical structure (compound of formula (I)):

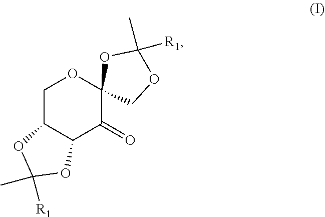

wherein

R₁ is a —CH₃-group.

The Shi catalyst is a very useful catalyst in specific epoxidation reactions (Shi epoxidation). The Shi epoxidation is a chemical reaction described as the asymmetric epoxidation of alkenes with the Shi catalyst. This reaction is thought to proceed via a dioxirane intermediate, generated from the catalyst ketone by oxone (potassium peroxymonosulfate). The addition of the sulfate group by the oxone facilitates the formation of the dioxirane by acting as a good leaving group during ring closure.

Due to the importance of this catalyst, there is always a need for the improved synthesis of this specific catalyst.

The Shi catalyst is usually synthesized starting from D-fructose, which is ketalised with 2,2′-dimethoxypropane (in acetone, in presence of a Brönsed acid, e.g. HClO₄ at 0° C.) followed by an oxidation with pyridinium chlorochromate (PCC). The overall yield is around 50%.

The goal of the present invention was to find a new process for the production of the Shi catalyst, which has higher yield, and which is safe and easy to handle.

The new process according to the present invention is a two-step process

The new and improved process starts from D-fructose (compound of formula (II)),

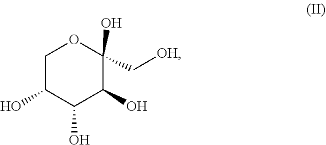 [SMILES: OC[C@@]1(O)OC[C@@H](O)[C@@H](O)[C@@H]1O]

which IUPAC name is (2R,3S,4R,5R)-2-(hydroxymethyl)tetrahydro-2H-pyran-2,3,4,5-tetraol is available commercially.

### Step (i)

In a first step (step (i)) D-fructose is reacted with a compound of formula (IV) (such as i.e. 2-methoxyprop-1-ene) in the presence of a solid acid catalyst.

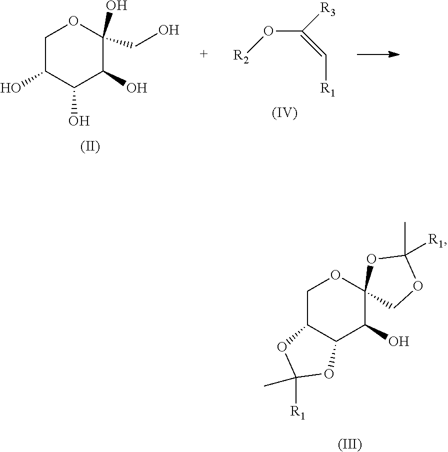

wherein

R₁ is a H or a C₁-C₃ alkyl group (preferably —H, —CH₃ or —CH₂CH₃), and

R₂ is a C₁-C₃ alkyl group (preferably —CH₃ or —CH₂CH₃), and R₃ is a H or a C₁-C₂ alkyl group (preferably —H or —CH₃).

The compounds of formula (III) are obtained in good yields.

A preferred compound of formula (III) is (3a′R,4S,7'S,7a'S)-2,2,2′,2′-tetramethyltetrahydro-spiro[[1,3]dioxolane-4,6′-[1,3]dioxolo[4,5-c]pyran]-7′-ol (also called bis-acetonide), which is the compound of formula (III) wherein R1 is —CH₃, is obtained in good yield.

It is clear from the reaction scheme that at least two Mol of the compound of formula (IV) is used when 1 Mol of D-fructose is used.

The reaction of step (i) is usually carried out in the presence of at least one solvent. Suitable solvents are polar solvents, such as ketones (such as acetone, diethylketone) or ethers (such as THF or 2-methyl-THF).

The reaction of step (i) is usually carried out at lower temperature. Suitable temperatures are between −5° C.-10° C.

As stated above an essential feature of step (i) is the presence of at least one solid acid catalyst.

These solid acid catalysts are functionalized ion-exchange resins. They are usually based on crosslinked polystyrene (by co-polymerisation of styrene and a few percent of divinylbenzene) and functionalized by acid groups such as sulfonic acid.

Suitable (and preferred) acid catalysts are i.e. Amberlyst® 15 (from Dow Chemicals) or p-TsOH polymer bounded.

The amount of the catalyst can vary. It is usually between 0.05-0.5 mol-eq (in view of D-fructose).

The reaction time of the reaction of step (i) is usually a few hours.

At the end of the reaction the compound of formula (III) is isolated.

The isolated product (compound of formula (III)) is then used in step (ii).

### Step (ii)

Step (ii) is the oxidation of the compound of formula (III) to the Shi catalyst (compound of formula (I)).

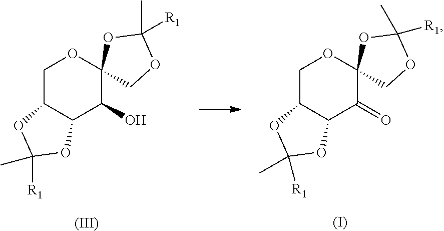

wherein

R₁ is a C₁-C₃ alkyl group (preferably —CH₃ or —CH₂CH₃).

This step (ii) is known from the prior art (i.e. from Ager et al, Organic Process & Development 2007, 11, 44-51).

The reaction of step (ii) is usually carried out in at least one solvent. Suitable solvents are polar aprotic solvents, such halogenated solvent and or diethoxymethane, which is used in the prior art.

The reaction of step (ii) is usually carried out in presence of at least one catalyst, which is usually a transition metal catalyst. Preferably the transition metal is ruthenium.

A very suitable catalysts is RuCl₃ x(H₂O)/NaIO₄.

The catalyst can be used in a ratio of up to 1:50 (catalyst to substrate).

The reaction of step (ii) is usually carried out at temperature of 20-60° C.

The compounds of formula (I) can be used for reaction as described in the prior art. These are (preferably) asymmetric epoxidation of olefins.

Therefore, the present invention also relates to the use of the compounds of formula (I) as epoxidation catalysts.

Furthermore, some of the compounds of the formula (I) are not known.

Therefore, the present invention also relates to compound of formula (Ia)

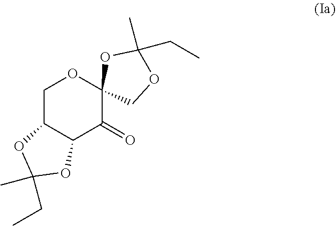

The following examples serve to illustrate the invention.

All parts and percentages are related to weight.

### EXAMPLES

### Example 1: Synthesis of Compound of Formula (III) (Step(i))

In a 200 ml flask 125 mMol D(−)fructose, 80 ml acetone and 0.2 eq Amberlyst 15 were stirred and cooled to 0° C.

Afterwards 280 mMol (2.236 eq) 2-methoxyprop-1-ene were solved in 40 ml acetone, transferred to a dropping funnel and added dropwise over 20 minutes to the reaction mixture at the same temperature (0° C.).

The temperature was maintained at 0-2° C. for 20 h.

After that, all fructose was reacted. Amberlyst 15 was filtered off. The pH was then adjusted to >7 (around pH 10) with 44% NaOHaq. The acetone was removed under vacuum on a rotary evaporator at 40° C. 25 ml Toluene were added and removed on the rotary evaporator to aid in the removal of the remaining traces of acetone. The residue (white slurry) was then dissolved in 100 ml toluene and washed with 30 ml saturated NaClaq and then 6 ml water. Aqueous phases one time extracted with toluene. Approximately half of the toluene was removed by vacuum on the rotary evaporator. 125 ml n-Heptane were added slowly to the warm solution. The resultant slurry was slowly cooled to <5° C. and filtered off.

The crude product (compound of formula (III) was washed with cold n-heptane and dried at 50° C. for 12 hours at 10-15 mbar to give 15.2 g of a product with a purity of 100% (GC area %), 58.4 mMol, yield 46.7%. The concentrated mother liquor contains further 17.2% (GC area %) of the product, yield 6.3%.

### Example 1: Synthesis of Compound of Formula (I) (Step(ii))

In a 100 ml flask 5.21 g (20 mMol) Bis-acetonide (compound of formula (III) obtained) was dissolved in 26 ml diethoxymethane. To this solution, 65 mg tetrabutylammonium bromide (99.9%, 0.01 eq, 0.20 mMol), 0.637 g K₂CO₃ (99.8%, 0.23 eq, 4.6 mMol), and 0.135 g RuCl3.H2O (0.03 eq, 0.60 mMol) were added.

A solution of 6.38 g NaIO4 (99.2%, 1.48 eq, 29.6 mMol) in 44 g water was prepared and added to the alcohol slowly, keeping the temperature below 40° C. by external cooling with an ice bath. After 1 hour at room temperature the reaction was completed. 1.3 ml (0.84 eq, 16.85 mMol) 2-propanol was added. The mixture was stirred for 30 minutes, followed by a filtration over dicalite and washed with ethyl acetate. The clear phases were separated after extraction. Aqueous phase one time extracted with ethyl acetate. The organic extracts were washed with Na2SO3aq 10% solution followed by NaClaq 10%. The ethyl acetate was removed on a rotary evaporator and the residue (5.34 g) was dissolved in ˜40 ml heptane under heating. The solution was slowly cooled to room temperature until a slurry was formed. It was then cooled to 0° C., and the ketone was isolated by filtration. The wet cake was washed with cold heptane and dried in a vacuum oven at 50° C. and 10-15 mbar.

4.51 g of white crystals of the compound of formula (I) with a purity of 94% by GC area % (82% yield) were obtained. The mother liquor was concentrated and a further 0.4 g. The residue is of the same quality=7.3% yield.

1. Process to produce compounds of formula (I) 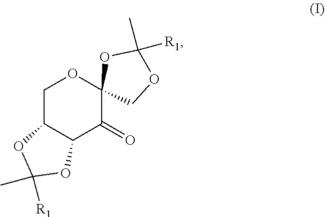
wherein in a first step (step (i) the compound of formula (II), 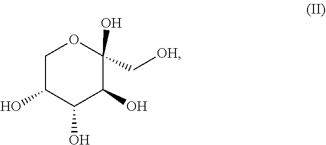 [SMILES: OC[C@@]1(O)OC[C@@H](O)[C@@H](O)[C@@H]1O]
is reacted with a compound of formula (IV) 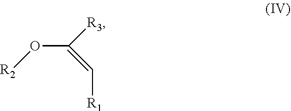
wherein
R₁ is H or a C₁-C₃ alkyl group (preferably —H, —CH₃ or —CH₂CH₃), and
R₂ is a C₁-C₃ alkyl group (preferably —CH₃ or —CH₂CH₃), and
R₃ is H or a C₁-C₂ alkyl group (preferably —H or —CH₃),
wherein R is a C₁-C₃ alkyl group in the presence of a solid acid catalyst and wherein the reaction product of step (ii), which is the compound of formula (III) 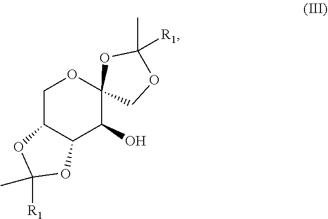
wherein
R₁ is the same as defined for the compound of formula (IV),
is oxidized to a compound of formula (I).

2. Process according to claim 1, wherein the reaction of step (i) is carried out in the presence of at least one solvent. (preferably in at least one polar solvent).

3. Process according to claim 1, wherein the reaction of step (i) is carried out at temperatures between −5° C.-10° C.

4. Process according to claim 1, wherein the solid acid catalyst are functionalized ion-exchange resins.

5. Process according to claim 1, wherein the amount of the catalyst in step (i) is between 0.05-0.5 mol-eq (in view of D-fructose).

6. Process according to claim 1, wherein the reaction of step (ii) is carried out in at least one solvent (preferably in at least one polar aprotic solvent).

7. Process according to claim 1, wherein the reaction of step (ii) is usually carried out in presence of at least one transition metal catalyst (preferably a ruthenium metal catalyst).

8. Process according to claim 1, wherein compound of formula (IV)
R₁ is H, and
R₂ is —CH₃, and
R₃ is —CH₃.

9. Process according to claim 1, wherein compound of formula (IV)
R₁ is —CH₂CH₃, and
R₂ is —CH₃, and
R₃ is H.

10. Compound of formula (Ia) [chemical structure]
